(19) United States
(12) Patent Application Publication
(10) Pub. No.: US 2002/0048596 A1
(43) Pub. Date: Apr. 25, 2002

(54) PREPARATION FOR THE TRANSPORT OF AN ACTIVE SUBSTANCE ACROSS BARRIERS

(76) Inventor: GREGOR CEVC, HEIMSTETTEN (DE)

Correspondence Address: DAVIDSON, DAVIDSON & KAPPEL, LLC, 485 SEVENTH AVENUE, 14TH FLOOR, NEW YORK, NY, 10018, US

(21) Appl. No.: 09/284,683

(22) PCT Filed: Oct. 17, 1996
(86) PCT No.: PCT/EP96/04526

(51) Int. Cl. A61K 9/127

(52) U.S. Cl. 424/450

(57) ABSTRACT

Preparation for the application of an active substance in the form of minute droplets especially liquid droplets with a membrane-type sheath of at least one or more layers of amphiphilic molecules or an amphiphilic carrier substance, especially for the transport of an active substance into and through natural barriers and constrictions such as skin and the like. The preparation has no point of solubilization or the preparation composition is at maximum permeation capacity far from solubilization point. The preparation contains at least two components whose solubility in suspending agents of the preparations, generally water, differs by at least a factor of 10.

The invention relates to new preparations for the administration of active ingredients in the form of very small liquid droplets, which can be suspended in a liquid medium, have a membrane-like sheath of one or a few layers of molecules, comprise an active ingredient and, in particular, are suitable for transporting the active ingredient through barriers, such as natural permeability barriers and constrictions in skin, mucous membranes, organs and the like.

Moreover, the invention relates to a method for the production of such preparations, especially for the non-invasive administration of active ingredients.

The administration of active ingredients frequently is limited by natural barriers, such as the skin, which prevent adequate introduction of active ingredients, since they are not sufficiently permeable to the active ingredients. For example, because of the permeability barrier of the skin, most current therapeutic agents must be administered perorally or parenterally (i.v., i.m., i.p.). Intrapulmonal and intranasal applications of aerosols, the use of rectal suppositories, the application of gels to mucous membranes, ocular preparations, etc. can be realized only at certain places and not with all active ingredients. The introduction of active ingredients into vegetable tissue is subject to even greater limitations because of the cuticular wax layers.

Non-invasive administrations of active ingredient preparations, which are suitable for penetrating such permeability barriers, would be advantageous in many cases. In man and animal, for example, a percutaneous administration of such preparations would protect the active ingredients administered against decomposition in the gastrointestinal tract and possibly result in a modified distribution of the agent in the body; it can affect the pharmacokinetics of the drug and permit a frequent as well as a simple non-invasive treatment (Karzel, K., Liedtke, R. K. (1989) Arzneim. Forsch./Drug Res. 39, 1487-1491). In the case of plants, improved penetration through or into the cuticle could lower the concentration of active ingredient required for the desired effect and, in addition, could significantly decrease contamination of the environment (Price, C. E. (1981) In: The Plant Cuticle (D. F. Cutler, K. L. Alvin, C. E. Price, Publisher), Academic, New York, pp.237-252).

Efforts to influence skin permeability by suitable measures have been discussed frequently (see, for example, Karzel and Liedtke, op. cit.). Especially worth mentioning are, for example, Jet injection (Siddiqui & Chien (1987) Crit. Rev. Ther. Drug. Carrier, Syst. 3, 195-208), the use of electrical fields (Burnette & Ongpipattanakul (1987) J. Pharm. Sci. 76, 765-773) or the use of chemical additives, such as solvents or surfactants. A long list of inactive ingredients, which were tested for the purpose of increasing the penetration of a water-soluble active ingredient (Nolaxon) into the skin, is contained, for example, in the work of Aungst et. al. (1986, Int. J. Pharm. 33, 225-234).

The best-known method for increasing penetration of active ingredient through the skin or mucous membrane is based on the use of penetration enhancers. Such penetration enhancers comprise nonionic materials (long-chain alcohols, surfactants, zwitterionic phospholipids), anionic materials (particularly fatty acids), cationic long-chain amines, sulfoxides, as well as various amino derivatives, and amphoteric glycinates and betaines. Nevertheless, the problem of the penetration of active ingredient into the skin has not yet been solved or not yet been solved satisfactorily.

An overview of the measures, which have been used for the purpose of increasing active ingredient penetration through plant cuticles, is summarized in the work of Prince (1981, op. cit.).

The penetration enhancers, which have previously exclusively been used occlusively, increase the ability to penetrate the permeability barrier of the skin or mucous membrane surface, in that they increase the fluidity of a portion of the lipids in this barrier. When chemical penetration enhancers were used, it has previously been customary to add these simply to a mixture containing the active ingredient; only in the case of human skin were additives sometimes also applied especially in the form of an organic solution. This form of administration was associated with the previously investigated and discussed principles of action of additives. In general, it was assumed that the increased penetration of the agent, on the one hand, is based on the softening (fluidization) of the skin (Golden et. al. (1987) J. Pharm. Sci. 76, 25-28). As a rule, the softening of the skin is associated with a destruction of the skin surface and its protecting barrier properties and consequently is undesirable. On the other hand, it was shown that some active ingredients permeate through the skin in the form of low molecular weight complexes with the additive molecules (Green et. al. (1988) Int. J. Pharm. 48, 103-111).

Proposals, deviating from these concepts, such as the epidermal use of lipid suspension, have brought about little improvement until now. Such suspensions typically contain vesicles or O/W or W/O emulsifiers.

The percutaneous use of carriers on a lipid basis, the liposomes (Patel, Bioch. Soc. Trans., 609^{th} Meeting, 13, 513-517, 1985, Mezei, M. Top. Pharm. Sci. (Proc. 45^{th} Int. Congr. Pharm. Sci. F.I.P.) 345-58 Elsevier, Amsterdam, 1985), which was discussed theoretically by several authors, was directed mainly at influencing the kinetics of the active ingredient. There was discussion of the use of conventional lipid vesicles, which pass through the skin extremely incompletely, if at all, as shown in this patent application. The use of liposomes, niosomes or other conventional lipid vesicles is therefore limited to external layers of the skin.

In a similar sense, the Japanese patent application JP 61/271204 A2 (86/271204) took up the use of liposomes by using hydroquinone glucosidal as a material, which increases the stability of the active ingredient.

The use of lipid vesicles carrying the active ingredient, together with a gel-forming agent, in the form of “transdermal patches” was proposed as an improvement in the WO 87/1938 A1. In this way, it was possible to prolong the period of action; however, the ability of the active ingredient to permeate was hardly increased. By the massive use of penetration-promoting polyethylene glycol and fatty acids together with lipid vesicles, Gesztes and Mezei (1988, Anesth. Analg. 67, 1079-1081) succeeded in attaining local analgesia with lidocaine-containing carriers, however, only after several hours of occlusive application and on a small scale.

Furthermore, carrier formulations were found, which are suitable for penetrating into and through permeability barriers. For example, it was possible for the first time to surpass the results of Gesztes and Mezei dramatically by a special formulation, which contained filtered, detergent-containing lipid vesicles (liposomes) with a declared optimum lipid/surfactant content of 1-40/1 and, in practice, generally of 4/1.

Furthermore, it was recognized that all such carriers, which are sufficiently elastic in order to be able to penetrate through the constriction of the barrier, such as of the skin, are suitable for penetrating into and through permeability barriers. This is so particularly if the carriers, after the application itself, build up a gradient at the permeability barrier, since in this case they tend to penetrate the permeability barrier spontaneously. In the DE 41 07 152 and DE 41 07 153 patent applications, carriers, which are referred to in the following as transfersomes, are described for the first time; they are useful for transporting active ingredients through almost any permeation barrier.

Transfersomes differ from the liposomes, previously described for topical use, and from other carriers used with respect to several basic properties. As a rule, transfersomes are much larger than conventional micelle-like carrier formulations and are therefore subject to different diffusion laws. For example, the permeability is not a linear function of the driving pressure, as it is in the case of liposomes, that is, in the case of transfersomes, the permeability, in contrast to liposomes or other known similar carrier systems, increases disproportionately or nonlinearly as the pressure increases. Furthermore, substances introduced through constrictions by means of transfersomes, can develop in man almost 100% of the maximum obtainable biological or therapeutic potential. For example, more than 50% and frequently more than 90% of the active ingredients, which have been applied percutaneously and packaged in transfersomes, regularly reach their site of destination in the body. These transfersomes, described in the EP 91 114 163 and PCT/EP 91/01596, contain a boundary-active substance, which corresponds up to 99 mole percent of the content and at least 0.1 mole percent of this substance, by means of which the solubilizing point of the droplets is attained.

The content of boundary-active substance, which brings about an optimized approximation of the solubilization limit of the transfersomes (that is, a content of boundary-active substance, which destabilizes the transfersomes completely), so that they are sufficiently elastic in order to be able to penetrate through constrictions in the barrier, such as those in the skin, was stated to be the decisive condition for the ability of the transfersomes to penetrate, which is greater than that of the liposomes or of similar known carriers.

For the formulation of such high-grade preparations capable of permeating, it would now be highly desirable not to be bound by the content ranges named.

It is therefore an object of the invention to indicate transfersomes for the administration of active ingredients, which transfersomes either do not have a solubilization point or are far removed from the solubilization point and permit the rapid and effective transport of active ingredients through barriers and constrictions.

It is furthermore an object of the invention to make available transfersomes for the transport of active ingredients through human, animal and vegetation barriers, which transfersomes make possible the improved availability of the active ingredient at the site of action.

It is furthermore an object of the invention to indicate a method for the preparation of such transfersomes for transporting active ingredients.

The distinguishing features of the independent claims serve to accomplish this objective.

Advantageous developments are given in the dependent claims.

Surprisingly, it was found that it is also possible to form transfersome preparations, which are suitable for the administration or transport of at least one active ingredient, especially for medical and biological purposes, into and through natural barriers and constructions, such as skin and the like, and have the form of liquid droplets, which can be suspended in a liquid medium and are provided with a membrane-like sheath of one or a few layers of amphiphilic carrier substance, the carrier substance comprising at least two amphiphilic components, which are physically and/or chemically different and differ in their solubility in the suspension medium of the transfersomes (usually water), by a factor of at least 10, if their content of solubilizing components amounts to less than 0.1 mole percent based on the content of these substances, for which the solubilizing point of the enveloped droplets is reached or the amphiphilic components are selected so that, independently of the concentration, there is no solubilization at all of the enveloped droplets.

The inventive preparations, referred to in the following once again as transfersomes, can be prepared from any amphiphilic components, which have sufficiently different solubilities. This condition is fulfilled, if the solubilities of the individual carrier components of the transfersome in the suspension medium differ at least by a factor of 10 (and of up to 10⁷). Fulfilling this condition ensures that the membrane-like sheath of the resulting transfersomes, under the influence of a gradient such as an intact natural barrier like the skin, has an increased deformability. This property enables the inventive transfersomes to penetrate through the constrictions in any permeability barriers.

The ability of the inventive preparations to permeate through constrictions is at least 0.001 percent and, preferably, more than 0.1 percent of the permeability of small, essentially unimpeded, permeated molecules.

According to present knowledge (but without having to be bound to a theoretical, scientific definition), the concept of solubility, as used here, refers to so-called true solutions. In any case, when a limiting concentration is reached, a solubility limit is observed, which is defined by the formation of a precipitate, the formation of crystals, the formation of suspensions or by the formation of molecular aggregates, such as micelles. For self-aggregating molecules, the solubility limit typically corresponds to the critical self-aggregation concentration (CAC). For molecules forming micelles, the solubility limit typically corresponds to the critical micelle concentration (CMC).

The inventive transfersomes differ appreciably from the previously described transfersomes. In particular, the transfersomes of the present application differ from known transfersomes owing to the fact that the transfersomes can be formed from combinations of any components, irrespective of their solubilizing capability.

Moreover, the stability of the inventive transfersomes is even better than that of the known transfersomes (see patent applications WO 92703122 and EP 475 160), since the transfersomes composition is not close to the solubilization point.

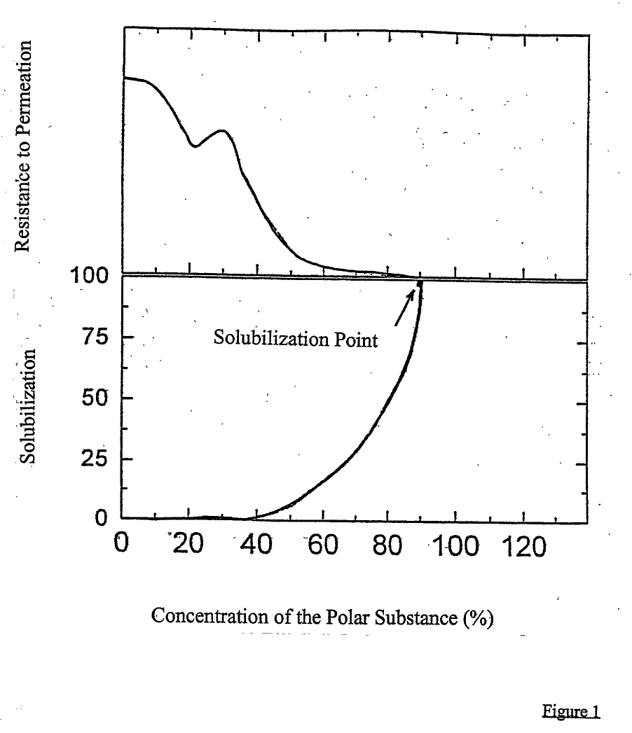

FIG. 1 shows the decrease in the permeation resistance at a barrier as a function of the concentration of boundary-active substance with respect to the approach to the solubilization point for transfersomes described in the state of the art (this solubilization point, however, not being reached).

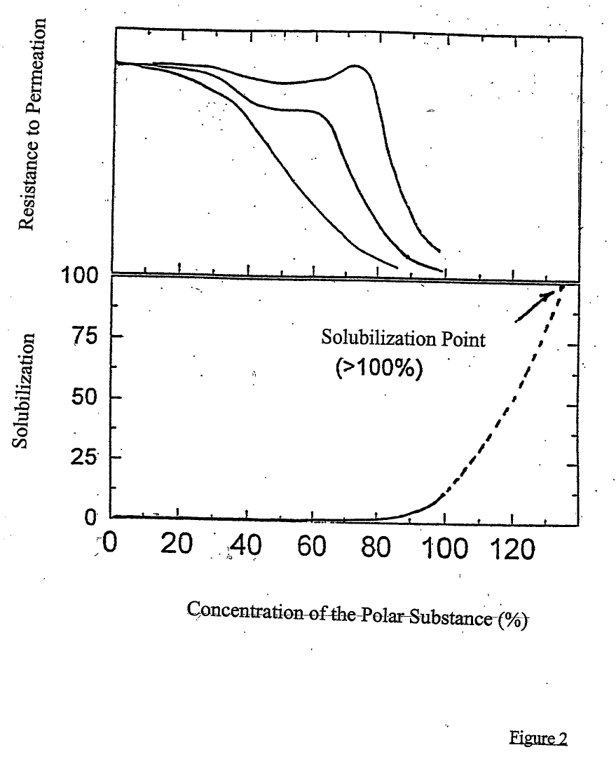

FIG. 2 shows, for inventive transfersomes, the decrease in the permeation resistance at a barrier as a function of the component concentration with respect to the approach to a theoretical solubilization point, which cannot be reached in practice.

FIG. 2 clearly shows that, for the component system of the inventive transfersomes, there is no solubilization point or the solubilization point is still far away when the maximum permeation capability is reached.

The inventive transfersomes accordingly open up an elegant, uniform and generally useful path for the transport of various active ingredients into or through permeability barriers. This newly discovered carrier for active ingredients is suitable for use in human and veterinary medicine, dermatology, cosmetics, biology, biotechnology, agricultural technology and in other areas.

A transfersome furthermore is distinguished by its ability to penetrate or diffuse under the action of a gradient through and/or into permeability barriers and, in so doing, transports materials, particularly active ingredients. This ability can easily be recognized and quantified owing to the fact that the curve, for which the permeation capability is plotted as a function of gradient, is not linear.

Pursuant to the invention, such a transfersome is composed of several to many molecules, which form a unit physicochemically, physically, thermodynamically and frequently functionally. The optimum transfersome size is a function of the barrier characteristics. It depends on the polarity (hydrophilicity), mobility (dynamics) and charge, as well as on the elasticity of the transfersome (surface). The size of a transfersome advantageously is between 10 and 10,000 nm.

Pursuant to the invention, transfersomes, preferably having a size of 50 to 10,000 nm, frequently of 75 to 400 nm and particularly of 100 to 200 nm, are used for dermatological applications.

For applications to plants, mostly relatively small transfersomes, predominantly with a diameter smaller than 500 nm, advisably are used.

The vesicle radius of the preparation droplets (transfersomes) is approximately 25 to 500, preferably 50 to 200 and particularly 80 to 180 nm.

For inventive transfersomes of any amphiphilic materials, preferably one or more components with a water solubility between 10⁻¹⁰ and 10⁻⁶ M and one or more components with a water solubility between 10⁻⁶ M and 10⁻³ M are combined. Alternatively, the amphiphilic components, which can be combined, can be assigned to one another also on the basis of their HLB values, the difference between the HLB values of the two components preferably amounting up to 10 and frequently being between 2 and 7 and, particularly, between 3 and 5.

The penetration capability of the inventive transfersomes can be determined by measurements, in which they are compared with reference particles or molecules. The reference particles used are clearly smaller than the constrictions in the barriers and accordingly have maximum permeation capability. Preferably, the permeation rate of transfersomes through a test barrier (P_{transfer.}), when the barrier itself is the site of the determination, should not differ by more than a factor of 10⁻⁵ to 10⁻³ from the permeation rate of the comparison materials P_{refer} (such as water). If a relatively uniform and slow transport of material through the barrier is desired, the given ratio should lie between 10⁻⁴ and 1. The permeation capability is at a maximum, when the ratio of P_{transfer.}/P_{refer.} is greater than 10⁻². This data refers to transfersomes, which are larger than the constrictions by more than a factor of 2 and less than a factor of 4. With increasing size difference between carrier and constrictions, that is, when the factor is greater than 4, the P_{transfer.}/P_{refer.} values can be correspondingly smaller.

Transfersomes of this application may consist of one or several components. Most frequently, a mixture of basic substances is used. Suitable basic substances comprise lipids and other amphiphilic substances, as well as hydrophilic liquids; these can be mixed with the active ingredient molecules in particular ratios, which depend on the choice of substances as well as on their absolute concentrations.

In general, the preparations contain at least two amphiphilic components of different solubility for forming a membrane-like sheath around an amount of droplet of a hydrophilic liquid, the active ingredient being contained in the membrane-like sheath, for example, a double membrane and/or in the hydrophilic liquid. The association between active ingredient and carrier may also take place at least partially only after the formation of transfersome-like droplets.

If the transfersomes inherently are not adequately deformable and their permeation capability is to be attained by the addition of boundary-active materials, the concentration of these materials corresponds to less than 0.1 mole percent of the amount, which would be required for solubilizating the transfersomes, or this solubilization is not attainable at all in the practically relevant concentration range.

The inventive transfersomes are useful for transporting active ingredients through almost any permeation obstacle, for example, for a percutaneous administration of a drug. They can transport water-soluble, amphiphilic or fat-soluble agents and, depending on their composition, on the amount applied and on their form, attain different depths of penetration. The special properties, which make a carrier out of a transfersome, can be attained by phospholipid-containing vesicles as well as by other amphiphilic aggregates. For example, a large proportion of active ingredient molecules can be carried not only into the barrier, for example, into the skin, but also through the barrier by means of such transfersomes and consequently become systemically active. For example, transfersomes carry polypeptide molecules through the skin 1000 times more efficiently than was previously possible with the help of permeation-promoting structureless materials.

### DEFINITIONS

Lipids:

In the sense of this invention, a lipid is any substance, which has properties like or similar to those of a fat. As a rule, it has an extended apolar group (the chain, X) and generally also a water-soluble, polar hydrophilic part, the head group (Y) and has the basic formula 1.

X—Yₙ (1)

wherein n is equal to or larger than zero. Lipids with n=0 are referred to as apolar lipids and lipids with n≧1 are referred to as polar lipids. In this sense, all amphiphilic substances, such as glycerides, glycerophospholipids, glycerophosphinolipids, glycerophosphonolipids, sulfolipids, sphingolipids, isoprenoid lipids, steroids or sterols and carbohydrate-containing lipids can generally be referred to as lipids.

A phospholipid is, for example, a compound of formula 2:

R—CH₂—CHR₂—CR₃H—POOₙ—O—R₄x G⁺ (2)

wherein n and R₄ have the meanings given under formula 2, R₁, R₂ cannot be hydrogen, OH or a short-chain alkyl group and R₃ generally is hydrogen or OH. Furthermore, R₄ is a short-chain alkyl group, substituted by a tri-short-chain alkylammonium group, such as a trimethylammonium group, or an amino-substituted short-chain alkyl group, such as 2-trimethylammonium ethyl group (cholinyl).

A lipid preferably is a substance of formula 2, wherein n 1, R₁ and R₂ are hydroxyacyl, R₃ is hydrogen and R4 is 2-trimethylammonium ethyl (the latter corresponds to the phosphatidyl choline head group), 2-dimethylammonium ethyl, 2-methylammonium ethyl or 2-aminoethyl (corresponding to the phosphatidyl ethanolamine head group).

Such a lipid is, for example, a natural phosphatidyl choline, which used to be called lecithin. It can be obtained from egg (rich in arachidonic acid), soybean (rich in C₁₈ chains), coconut (rich in saturated chains), olives (rich in monounsaturated chains), saffron (safflower) and sunflowers (rich in n-6 linoleic acid), linseed (rich in n-3 linolenic acid), from whale fat (rich in monounsaturated n-3 chains), from primrose or primula (rich in n-3 chains). Preferred, natural phosphatidyl ethanolamines (used to be called cephalins) frequently originate from egg or soybeans.

Furthermore, synthetic phosphatidyl cholines (R₄ in formula 2 corresponds to 2-trimethylammonium ethyl), synthetic phosphatidyl ethanolamines (R₄ is 2-aminoethyl), synthetic phosphatid acids (R₄ is a proton) or its ester (R₄ corresponds, for example, to a short-chain alkyl, such as methyl or ethyl), synthetic phosphatidyl serines (R₄ is L- or D-serine), or synthetic phosphatidyl (poly)alcohols, such as phosphatidyl inositol, phosphatidyl glycerol (R₄ is L- or D-glycerol) are preferred as lipids, wherein R₁ and R₂ are identical acyloxy groups, such as lauroyl, oleoyl, linoyl, linoleoyl or arachinoyl, such as dilauroyl, dimyristoyl, dipalmitoyl, distearoyl, diarachinoyl, dioleoyl, dilinoyl, dilinolenyl, dilinoloyl, dilinolinoyl, dilininolenoyl or diarachinoyl phosphatidyl choline or ethanolamine, or various acyl groups, such as R₁=palmitoyl and R=oleoyl, such as 1-palmitoyl-2-oleoyl-3-glycerophosphocholine, or various hydroxyacyl groups, such as R₁=hydroxypalmitoyl and R₄=oleoyl etc. Moreover, R₁ can represent alkenyl and R₂ identical hydroxyalkyl groups, such as tetradecylhydroxy or hexadecylhydroxy, for example, in ditetradecyl or dihexadecylphosphatidyl choline or ethanolamine, R₁ can represent alkenyl and R₂ hydroxyacyl, such as a plasmalogen (R₄ trimethylammonium ethyl), or R₁ can be acyl, such as lauryl, myristoyl or palmitoyl and R₂ can represent hydroxy as, for example, in natural or synthetic lysophosphatidyl cholines or lysophosphatidyl glycerols or lysophosphatidyl ethanolamines, such as 1-myristoyl or 1-palmitoyllysophosphatidyl choline or -phosphatidyl ethanolamine; frequently, R₃ represents hydrogen.

A lipid of formula 2 is also a suitable lipid within the sense of this invention. In formula 2, n=1, R₁ is an alkenyl group, R₂ is an acylamido group, R₃ is hydrogen and R₄ represents 2-trimethylammonium ethyl (choline group). Such a lipid is known under the name of sphingomyelin.

Suitable lipids furthermore are a lysophosphatidyl choline analog, such as 1-lauroyl-1,3-dihydroxypropane-3-phosphoryl choline, a monoglyceride, such as monoolein or monomyristin, a cerebroside, ceramide polyhexoside, sulfatide, sphingoplasmalogen, a ganglioside or a glyceride, which does not contain a free or esterified phosphoryl or phosphono or phosphino group in the 3 position. An example of such a glyceride is diacylglyceride or 1-alkenyl-1-hydroxy-2-acyl glyceride with any acyl or alkenyl groups, wherein the 3-hydroxy group is etherified by one of the carbohydrate groups named, for example, by a galactosyl group such as a monogalactosytl glycerin.

Lipids with desirable head or chain group properties can also be formed by biochemical means, for example, by means of phospholipases (such as phospholilpase A1, A2, B, C and, in particular, D), desaturases, elongases, acyl transferases, etc., from natural or synthetic precursors.

Furthermore, a suitable lipid is any lipid, which is contained in biological membranes and can be extracted with the help of apolar organic solvents, such as chloroform. Aside from the lipids already mentioned, such lipids also include, for example, steroids, such as estradiol, or sterols, such as cholesterol, β-sitosterol, desmosterol, 7-keto-cholesterol or β-cholestanol, fat-soluble vitamins, such as retinoids, vitamins, such as vitamin A1 or A2, vitamin E, vitamin K, such as vitamin K1 or K2 or vitamin D1 or D3, etc.

The less soluble amphiphilic components comprise or preferably comprise a synthetic lipid, such as myristoleoyl, palmitoleoyl, petroselinyl, petroselaidyl, oleoyl, elaidyl, cis- or trans-vaccenoyl, linolyl, linolenyl, linolaidyl, octadecatetraenoyl, gondoyl, eicosaenoyl, eicosadienoyl, eicosatrienoyl, arachidoyl, cis- or trans-docosaenoyl, docosadienoyl, docosatrienoyl, docosatetraenoyl, lauroyl, tridecanoyl, myristoyl, pentadecanoyl, palmitoyl, heptadecanoyl, stearoyl or nonadecanoyl, glycerophospholipid or corresponding derivatives with branched chains or a corresponding dialkyl or sphingosin derivative, glycolipid or other diacyl or dialkyl lipid.

The more soluble amphiphilic components(s) is/are frequently derived from the less soluble components listed above and, to increase the solubility, substituted and/or complexed and/or associated with a butanoyl, pentanoyl, hexanoyl, heptanoyl, octanoyl, nonanoyl, decanoyl or undecanoyl substituent or several, mutually independent, selected substituents or with a different material for improving the solubility.

A further suitable lipid is a diacyl- or dialkyl-glycerophosphoethanolamine azo polyethoxylene derivative, a didecanoylphosphatidyl choline or a diacylphosphoolligomaltobionamide.

Within the sense of this invention, any other substance (such as a poly- or oligoamino acid), which has a slight or at least regionally a slight solubility in polar materials, is regarded as a lipid.

All surfactants and asymmetric, and therefore amphiphilic molecules or polymers, such as some oligocarbohydrates and polycarbohydrates, oligopeptides and polypeptides, oligonucleotides and polynucleotides, many alcohols or derivatives of such molecules belong to this category.

The polarity of the “solvents”, surfactants, lipids or active ingredients depends on the effective, relative hydrophilicity/hydrophobicity of the respective molecule. However, it also depends on the choice of other system components and boundary conditions in the system (temperature, salt content, pH, etc.). Functional groups, such as double bonds in the hydrophobic group, which weaken the hydrophobic character of this group, increase the polarity; extensions of or bulky substituents in the hydrophobic group, such as in the aromatic group, lower the polarity of a substance. Charged or highly polar groups in the head group, while the hydrophobic chain remains the same, normally contribute to a higher polarity and solubility of the molecules. Direct bonds between the lipophilic and/or amphiphilic system components have the opposite action.

In particular, all compounds named in the EP patent application 475 160 as being boundary active, are suitable as highly polar substances. The disclosure of this patent application is herewith explicitly referred to.

ACTIVE INGREDIENTS:

The inventive transfersomes are suitable for the administration of the most different active ingredients, particularly, for example, for therapeutic purposes. For example, inventive preparations may contain, in particular, all the active ingredients named in the EP patent application 475 160.

Furthermore, inventive preparations may contain, as active ingredient, an adrenocorticostatic agent, a β-adrenolytic agent, an androgen or antiandrogen, an anti-parasitic, anabolic, anesthetic or analgesic, analeptic, anti-allergic, anti-arrhythmic, anti-arteriosclerosis, anti-asthmatic and/or bronchospasmolytic agent, an antibiotic, an anti-depressive and/or anti-psychotic agent, an anti-diabetic agent, an antidote, an anti-emetic, anti-epileptic, anti-fibrinolytic, anti-convulsive or anti-cholinergic agent, an enzyme, coenzyme or a corresponding inhibitor, an antihistamine, an antihypertensive drug, a biological activity inhibitor, an anti-hypotensive drug, an anticoagulant, an anti-mycotic or antimyasthenic agent, an active ingredient against Parkinson's or Alzheimer's disease, an anti-phlogistic, anti-pyretic or anti-rheumatic agent, an antiseptic, a respiratory analeptic or stimulating agent, a broncholytic, cardiotonic or chemotherapeutic agent, a coronary dilator, a cytostatic agent, a diuretic, a ganglion blocker, a glucocorticoid, a therapeutic agent for influenza, a hemostatic or hypnotic agent, immunoglobulin or fragment or a different immunological or receptor substance, a bioactive carbohydrate (derivative), a contraceptive, a migraine agent, a mineral corticoid, a morphine antagonist, a muscle relaxant, a narcotic, a neural or CNS therapeutic agent, a nucleotide or polynucleotide, a neuroleptic agent, a neuron transmitter or a corresponding antogonist, a peptide (derivative), an ophthalmic agent, a (para)-sympathicomimetic or (para)-sympathicolytic agent, a protein (derivative), a psoriasis/neurodermatitis agent, a mydriatic agent, a mood elevator, a rhinological agent, a sleeping draft or its antagonist, a sedative, a spasmolytic, tuberculostatic or urological agent, a vasoconstrictor or dilator, a virostatic agent or a wound-healing agent or several such agents.

Preferably, the active ingredient is a non-steroidal anti-inflammatory drug, such as diclofenac, ibuprofen or a lithium, sodium, potassium, cesium, rubidium, ammonium, monomethyl, dimethyl, trimethylammonium or ethylammonium salt thereof.

Moreover, the inventive preparations may contain, as active ingredient, a growth-regulating substance for living beings, a biocide, such as an insecticide, pesticide, herbicide, fungicide or an allurement, particularly a pheromone.

As less polar components, inventive preparations may contain a physiologically compatible lipid, preferably from the class of phospholipids and especially from the class of phosphatidyl cholines, the active ingredient, for example, ibuprofen, diclofenac or a salt thereof, being the more soluble component, optionally with the addition of less than 10% by weight, based on the total composition of the preparation of a further soluble component and the concentration of the more soluble component(s) typically being between 0.01% by weight and 15% by weight, preferably between 0.1% and 10% by weight and particularly between 0.5% by weight and 3% by weight, and the total lipid concentration being between 0.005% by weight and 40% by weight and preferably between 0.5% by weight and 15% by weight and especially between 1% by weight and 10% by weight.

Inventive preparations additionally may comprise consistency modifiers, such as hydrogels, antioxidants such as probucol, tocopherol, BHT, ascorbic acid, desferroxamine and/or stabilizers such as phenol, cresol, benzyl alcohol, etc.

Unless specified otherwise, all the substances indicated, surfactants, lipids, active ingredients or additives with one or more chiral carbon atoms can be used either as racemic mixtures or as optically pure enantiomers.

PRINCIPLE OF ACTION:

In the case of permeation barriers, the transport of the active ingredients can be accomplished by those transfersomes, which satisfy the following basic criteria:

The transfersomes shall sense or build up a gradient, which drives them into or over the barrier, for example, from the body surface into and under the skin, from the leaf surface into the interior of the leaf, from one side of the barrier to the other;

The resistance to permeation, which the transfersomes sense in the barrier, shall be as small as possible in comparison to the driving force;

The transfersomes shall be able to permeate into and/or through the barrier, without at the same time losing the enclosed active ingredients in an uncontrolled manner.

Furthermore, the transfersomes shall permit the distribution and effects of the active ingredient and the course of action as a function of time to be controlled. If necessary, they shall also be able to bring the material into the depth of the barrier and beyond the barrier and/or to catalyze such a transport. Last but not least, the transfersomes shall have an effect on the range and depths of action, as well as, in favorable cases, the nature of the cells, the tissue parts, the organs or the system sections, which are reached or treated.

In a first respect, the chemical gradients come into consideration for the biological applications. Particularly suitable are the physicochemical gradients, such as the (de)hydration pressure (moisture gradient) or a concentration difference between the site of application and the site of action; however, electrical or magnetic fields, as well as thermal gradients are of interest in this respect. For technical applications, the hydrostatic pressure applied or an existing pressure difference is furthermore of importance.

In order to fulfill the second condition, the transfersomes must be sufficiently “liquid” on the microscopic scale, that is, they must have a sufficiently high mechanical elasticity and deformability and a sufficiently low viscosity; only then can they pass through the constrictions within the permeability barrier.

Understandably, the resistance to permeation decreases with carrier size. However, the driving force frequently also depends on the carrier size; if the pressure is independent of size, this force typically decreases with size. For this reason, the transfer coefficient is not a simple function of size and frequently has a maximum, which depends on the choice of carrier and active ingredient.

Furthermore, the choice of carrier substance, active ingredients and additives, as well as the amount or concentration of carrier applied play a role. A low dosage generally leads to a surface treatment. At the same time, materials of low water solubility generally remain in the apolar region of the permeability barrier (for example, in the membranes of the epidermis). Readily soluble active ingredients, which diffuse easily out of the carriers, may have a distribution different from that of the carrier. For such materials, the permeability of the transfersome membrane is also of importance. Substances, which tend to cross over from the carriers into the barrier, lead to a locally variable carrier composition, etc. These relationships should be considered and taken into consideration before any application. When searching for conditions, under which simple carrier vesicles become transfersomes, the following rule of thumb can be used:

To begin with, two or more amphiphilic components are combined, which differ in their solubility in the intended suspension medium of the transfersomes, usually water or a different polar, generally aqueous medium by a factor of 10 to 10⁷, preferably of 10² to 10⁶ and especially of 10³ to 10⁵, the less soluble component having a solubility of 10⁻¹⁰ to 10⁻⁶ and the more soluble component a solubility of 10⁻⁶ to 10⁻³ M. The solubility of the corresponding components, if not known from general, conventional reference works, can be determined, for example, by conventional methods of determining the saturation limit.

As a next step up, the carrier composition or concentration of the components in the system is adapted, so that the vesicles are sufficiently stable as well as adequately deformable, and therefore have appropriate permeation capability. In this application, stability is understood to be mechanical “coherence” as well as the fact that the substance content and, in particular, the active ingredient content of the carrier composition does not change or does not change significantly during the transport and particularly not during the permeation process. The position of the optimum sought depends on the components selected.

Finally, the system parameters are optimized, taking into consideration the application methods and the objectives aimed for. For a rapid action, a high permeation capability is required; for a slow release of active ingredient, a gradual penetration of the barrier and a correspondingly adjusted membrane permeability are advantageous; for action at a depth, a high dose is advisable and for as wide a distribution as possible, a carrier concentration that is not too high.

The content of amphiphilic components is adjusted, in particular, so that the ability of the transfersomes preparation to permeate through constrictions is at least 0.001 percent of the permeability of small molecules (for example, water). The ability of the inventive transfersomes to penetrate can be determined by means of measurements, in which the transfersomes are compared with reference particles or molecules. The reference particles used are clearly smaller than the constrictions in the barrier and thus have maximum permeation capability. Preferably, the difference between the transfersome permeation rate through a test barrier (P_{transfer.}) and the permeation rate of the comparison materials (P_{refer.}) (such as water) should not be greater than a factor of 10⁻⁵ to 10⁻¹ when the barrier itself is the site of the determination.

In this application, relevant properties of the transfersomes as carriers for the lipid vesicles are discussed. Most of the examples refer, by way of example, to phospholipids as carrier. However, the general validity of the conclusions is not limited to this class of carriers or these molecules. The lipid vesicle examples merely illustrate the properties, which are required for penetration through the permeability barriers, such as the skin, for example. The same properties also make possible the transport of a carrier through the animal or human epidermis, mucous membranes, plant cuticles, inorganic membranes, etc.

The probable reason for the spontaneous permeation of transfersomes through the “pores” in the layer of corneal corpuscles is the fact that one side of these pores ends in an aqueous compartment, the subcutaneous tissue; for this permeation, the transfersomes are driven by osmotic pressure. Alternatively, however, an external pressure, such as a hydrostatic or an electro-osmotic pressure can be applied additionally.

Depending on the amount of vesicles, the lipid vesicles can reach as far as the subcutaneous tissue after a percutaneous application. The active ingredients, depending on the size, composition and formulation of the carrier or agents, are released locally, accumulated proximally or passed on over lymph or blood vessels and distributed over the body.

It is sometimes appropriate to adjust the pH of the formulation immediately after the production or immediately before use. Such an adjustment is intended to prevent the destruction of the components of the system and/or of the active ingredient carriers under the initial pH conditions and to ensure the physiological compatibility of the formulation. For the neutralization, physiologically compatible acids or bases and buffer solutions with a pH of 3 to 12, preferably of 5 to 9 and especially of 6 to 8, depending on the purpose and site of the application, are generally used. Physiologically compatible acids are, for example, dilute aqueous mineral acids, such as dilute hydrochloric acid, sulfuric acid or phosphoric acid, or organic acids such as alkane carboxylic acids like acetic acid. Physiologically compatible alkalis are, for example, dilute sodium hydroxide solution, appropriately ionized phosphoric acid, etc.

The preparation temperature is normally adapted to the substances used and, for aqueous preparations, usually is between 0° and 95° C. Preferably, the temperature ranges from 18° to 70° C.; for lipids with fluid chains, the temperature range preferably is between 15° and 55° C. and, for lipids with ordered chains, between 45° and 60° C. Other temperature ranges are possible for non-aqueous systems or preparations, which contain cooling or heating preservatives, or which are prepared in situ.

If required by the sensitivity of the components of the system, the formulations can be stored cool (for example, at 4° C.). They can also, however, be prepared and stored under the atmosphere of an inert gas, such as nitrogen. The shelf life can be increased further by using substances without multiple bonds, as well as by drying and using dry substance, which is dissolved and worked up only on the spot. In particular, the transfersomes-like droplets can be prepared from a concentrate or a lyophilisate shortly before use.

In most cases, the carriers are applied at room temperature. Use at lower temperatures or at higher temperatures, with synthetic substances at even higher temperatures, is entirely possible.

A transfersomes suspension can be produced by means of supplying mechanical, thermal, chemical or electrical energy. For example, the preparation of a transfersome can be based on homogenizing or stirring.

The formation of transfersome-like droplets can be brought about by filtration. The filter material, which can be used for this purpose, should have a pore size of 0.01 to 0.8 μm, especially of 0.05 to 0.3 μm and particularly of 0.08 to 0.15 μm. Optionally, several filters can be arranged in series.

The preparations can be prepared in advance or at the site of use, as described, for example, in P 40 26 833.0-43 or by means of several examples in the handbook “Liposomes” (G. Gregoriadis, published by CRC press, Boca Raton, Fla., volumes 1 to 3, 1987), in the book “Liposomes as Drug Carriers” (G. Gregoriadis, published by John Wiley & Sons, New York, 1988), or in the laboratory handbook “Liposomes. A Practical Approach” (R. New, Oxford Press, 1989). If necessary, an active ingredient suspension can be diluted or concentrated immediately before use, for example, (by ultracentrifugation or ultrafiltration) or mixed with further additives. For this, however, the possibility, that the optimum for the carrier permeation will be shifted, must be excluded or taken into consideration.

The transfersomes of this application are suitable as carriers of lipophilic materials, such as fat-soluble biological active ingredients, therapeutic agents and poisons, etc.; their use in connection with amphiphilic, water-soluble substances is also of great practical value, particularly when their molecular weight is greater than 1,000.

The transfersomes furthermore can contribute to stabilizing hydrolysis-sensitive materials and to make an improved distribution of agents in the sample and at the site of application possible, as well as to ensuring a more advantageous temporal course of the action of the active ingredient. The basic substance, of which the transfersomes consist, can itself have an advantageous effect. The most important carrier property, however, is to enable material to be transported into and through the permeability barrier.

Pursuant to the invention, the formulations described are optimized for topical application at or in the vicinity of permeability barriers. The application on the skin or on the plant cuticle ought to be particularly interesting. (However, they are also well suited for oral (p.o.) or parenteral (i.v., i.m. or i.p.) administration, particularly if the composition of the transfersome is selected so that losses at the site of administration are small.) Substances or components, which are decomposed preferentially at the site of application, taken up particularly readily or diluted, are particularly valuable in the last respect, depending on the intended use.

In the medical area, preferably up to 50, frequently up to 10, especially fewer than 2.5 or even fewer than 1 mg of carrier substance are applied per cm² of skin surface; the optimum amount depends on the carrier composition, the depth or duration of action aimed for as well as on the site of application. In the agrotechnical area, the amounts applied typically are lower and frequently less than 0.1 g/m².

In particular, the total content of amphiphilic substance to be applied on human or animal skin ranges from 0.01 to 40% by weight of the transfersome, preferably from 0.1 to 15% by weight and especially from 1 to 10% by weight.

For application on plants, the total content of amphiphilic substance ranges from 0.000001 to 10% by weight, preferably from 0.001 to 1% by weight and especially from 0.01 to 0.1% by weight.

Depending on the application aimed for, the formulations, pursuant to the invention, may also contain suitable solvents up to a concentration, which is determined by the respective physical (no solubilization or no shift in the optimum worth mentioning), chemical (no effect on the stability), or biological or physiological (few undesirable side effects) compatibility.

Preferably, unsubstituted or substituted hydrocarbons, such as halogenated, aliphatic, cycloaliphatic, aromatic or aromatic aliphatic hydrocarbons, such as benzene, toluene, methylene chloride or chloroform, alcohols, such as methanol or ethanol, butanol, propanol, pentanol, hexanol or heptanol, dihydroxypropane, erythritol, low molecular weight alkane carboxylate esters, such as alkyl acetates, ethers, such as diethyl ether, dioxane or tetrahydrofuran, or mixtures of these solvents come into consideration.

Overviews of the lipids and phospholipids which, in addition to those named above, are suitable for use in the sense of this application, are contained in ‘Form and Function of Phospholipids’ (Ansell & Hawthorne & Dawson, publisher), ‘An Introduction to the Chemistry and Biochemistry of Fatty Acids and Their Glycerides’ by Gunstone and in other review works. The lipids and surfactants mentioned, as well as other boundary-active materials, which come into consideration, and their manufacture are known. A survey of the commercially obtainable polar lipids, as well as of the trademarks, under which they are sold by manufacturing companies, is given in the yearbook ‘McCutcheon's Emulsifiers & Detergents’, Manufacturing Confectioner Publishing Co. A topical list of the pharmaceutically acceptable active ingredients is given, for example, in the ‘Deutschen Arzneibuch’ (German Pharmacopoeia) (and the respective annual edition of the ‘Rote Liste’), furthermore also in the British Pharmaceutical Codex, the European Pharmacopoeia, the Farmacopoeia Ufficiale della Republica Italiana, the Japanese Pharmacopoeia, the Dutch Pharmacopoeia, the Pharmacopoeia Helvetica, the Pharmacopee Francaise, The United States Pharmacopoeia, The United States NF, etc. A detailed list of enzymes, suitable pursuant to the invention, is contained in the volume ‘Enzymes’, 3^{rd} Edition (M. Dixon and E. C. Webb, Academic, San Diego, 1979) and topical new developments can be found in the ‘Methods in Enzymology’ series. Sugar-recognizing proteins, which are of interest in connection with this invention, are described in the book ‘The Lectins: Properties, Functions and Applications in Biology and Medicine’ (I. E. Liener, N. Sharon, I. T. Goldstein, Eds., Academic, Orlando, 1986) as well as in topical technical publications; agrotechnically interesting substances are listed in ‘The Pesticide Manual’ (C. R. Worthing, S. B. Walter, Eds., British Crop Protection Council, Worcestershire, England, 1986, for example, 8^{th} edition) and in ‘Wirkstoffe in Pflanzenschutz und Schädlings-bekampfung’ (Active Ingredients in Plant Protection and Pest Control), published by the Industrie-Verband Agrar (Frankft); commercially obtainable antibodies are listed in the ‘Linscott's Directory’ catalog, the most important neuropeptides are listed in ‘Brain Peptides’ (D. T. Krieger, M. J. Brownstein, J. B. Martin, Eds., John Wiley, New York, 1983), corresponding supplemental volumes (such as 1987) and other technical publications.

Manufacturing techniques for liposomes, which are predominantly also suitable for the manufacture of transfersomes, are described in ‘Liposome Technology’ (Gregoriadis, Ed., CRC Press) or in older reference works, such as ‘Liposomes in Immunobiology’ (Tom & Six, Eds., Elsevier), in ‘Liposomes in Biological Systems’ (Gregoriadis & Allison, Eds., Willey), in ‘Targeting of Drugs’ (Gregoriadis & Senior & Trouet, Plenum), etc., as well as in the relevant patent literature.

The stability and permeation capability of transfersomes can be determined by filtration, if necessary under pressure, through a fine-pored filter or through otherwise controlled mechanical whirling up, shearing or comminuting.

The following examples illustrate the invention without limiting it. Temperatures are given in ° C., carrier sizes in nanometers, pressures in pascals and other quantities in conventional SI units.

Unless otherwise stated, ratios and percentages are molar and the measurement temperature is about 21° C.

### EXAMPLES 1-4

Summary:

| | | |
|---|---|---|
| 0-500 mg | phosphatidyl choline from soybeans | |
| | CMC = 10⁻⁷M (approx. 98% PC = SPC) | |
| 0-500 mg | distearoyl glycerophosphoethanolamine | CMC = 10⁻⁵M |
| | triazopolyethylene glycol (5000) | |
| 4.50 ml | buffer, pH 7.3 | |

Preparation:

Mixtures of SPC (molecular weight assumed to be 800 Da) with increasing amounts of 0, 30 and 40 mole percent DSPE-PEG (molecular weight assumed to be 5800 Da) and pure DSPE-PEG liposomes not containing any SPC are prepared. Subsequently, the mixtures obtained were dissolved in a chloroform/ methanol solution. After that, the lipid solution is transferred to a round-bottom flask. After removal of the solvent in a rotary evaporator, a thin lipid film remains behind on the wall of the flask. This film is dried further under vacuum (10 Pa), subsequently hydrated by addition of buffer and suspended by mechanical stirring. A cloudy suspension is obtained which, as a rule, is very viscous. The size of the particles in the resulting suspension is determined by means of dynamic light scattering as well as by means of microscopy. In all cases, the particle size observed is always greater than 0.5 μm. Therefore, for the mixtures investigated, micelle formation and consequently also solubilization can be excluded by means of dynamic light scattering.

The liposomes for the comparison experiments are prepared from a pure phosphatidyl choline by a similar method.

Determination of the Permeation Capability of the Carrier:

A carrier suspension is driven under an externally applied pressure through the constrictions in an artificial permeation barrier. The amount of material passing in unit time through the constriction, is determined volumetrically or gravimetrically. From the total area (application area of the material), the (driving) pressure, the time and the amount of penetrate, the permeation capability (P) of the suspension in the system investigated is calculated as follows:

$$P = \frac{amount of permeate}{time \times area \times driving pressure}$$

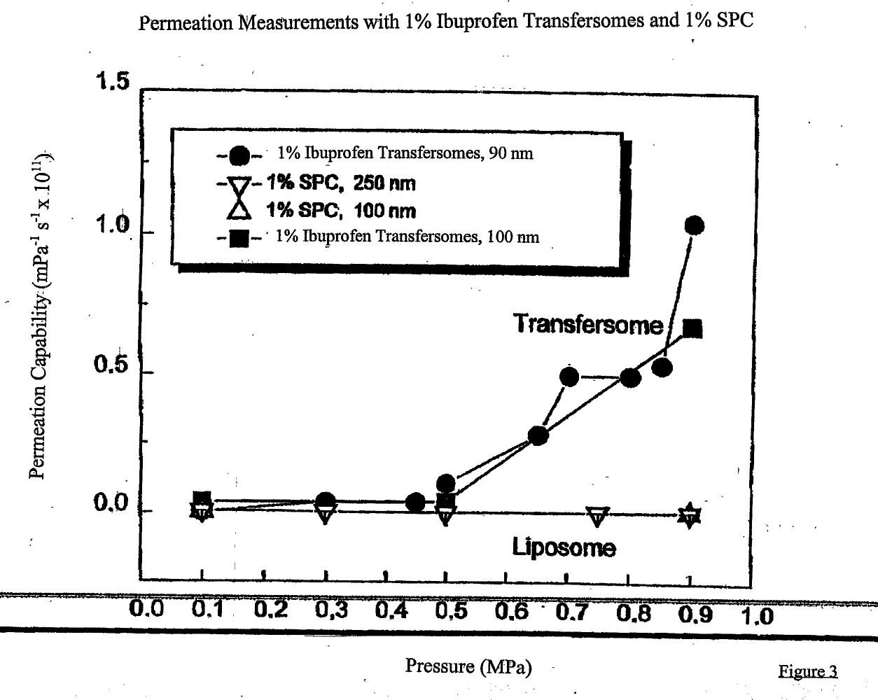

The measurement is repeated independently for several pressures. The relative dependence of the permeation capability, which is a measure of the carrier deformability, is calculated from the results of such measurements as a function of mechanical stress or pressure. The value for a hydrated, 1% solution, containing pure SPC, is approximately>0.01 μL/MPa/s/cm² at a pressure of 0.3 MPa (see FIG. 3).

The permeation capability measurement for such experimental series is carried out at 62° C., so as to ensure that both lipids exist as liquid phases.

The results of such a series of measurements for Examples 1 to 4 are shown in Table 1. Table 1 shows that, as the driving pressure increases, the permeation capability greatly increases, but not linearly and, at high droplet loads (0.7 MPa), is several orders of magnitude higher than the value, which results at a lower load (0.3 MPa). Such a pronounced nonlinear relationship, however, arises exclusively (in the sense of a difference criterion) for transfersomes and not for liposomes. It is clearly evident from FIG. 3 that the value of the permeation capability is several orders of magnitude lower for liposomes than for transfersomes. This difference in permeation capability between transfersomes and liposomes clearly shows that the penetration capability for transfersomes is significantly increased over that for liposomes.

TABLE 1

| | | Permeation | | |
|---|---|---|---|---|
| Sample Final Size | Pressure | Capability | Initial Size | |
| Description | (MPa) | (μl/MPa/sec/cm²) | (nm) | (nm) |
| SPC/DSPE - PEG | 0.7 | 22.3 | 225.7 | 92.5 |
| 70/30 mole % | 0.6 | 18.7 | | 94.5 |
| 10% lipid solution | 0.5 | 10.9 | | 96.1 |
| rehydrated | 0.4 | 2.8 | | 96.1 |
| sample | 0.3 | 0.007 | | 100.5 |
| SPC/DSPE - PEG | 0.7 | 12.2 | 217.3 | 96.3 |
| 60/40 mole % | 0.6 | 13.2 | | 100.7 |
| 10% Lipid solution | 0.5 | 12.2 | | 120 |
| Rehydrated | 0.4 | 3.39 | | 99.1 |

### EXAMPLES 5-6

Composition:

| | |
|---|---|
| 410.05 mg, 809.25 mg | phosphatidyl choline from soy beans |
| | (purer than 95%) CMC = 10⁻⁷M |
| 289.95 mg, 190.75 mg | didecanoyl phosphatidyl choline |
| | CMC ∞ 10⁻⁶ |
| 7 ml, 10 ml | buffer, pH 7.3 |

Preparation:

The respective lipid content is selected so that both lipid components are present in a molar ratio of 1:1 or 3:1 in the final formulation. The appropriate amounts of phosphorlipid are weighed into a 50 ml round-bottom flask and dissolved in each case in 1 ml of 1:1 chloroform/methanol. After removal of the solvent in the rotary evaporator, a suspension of the film is obtained as described in Examples 1 to 4 and has carriers with an average radius of approximately 450 nm.

Determination of the Carrier Permeation Capability

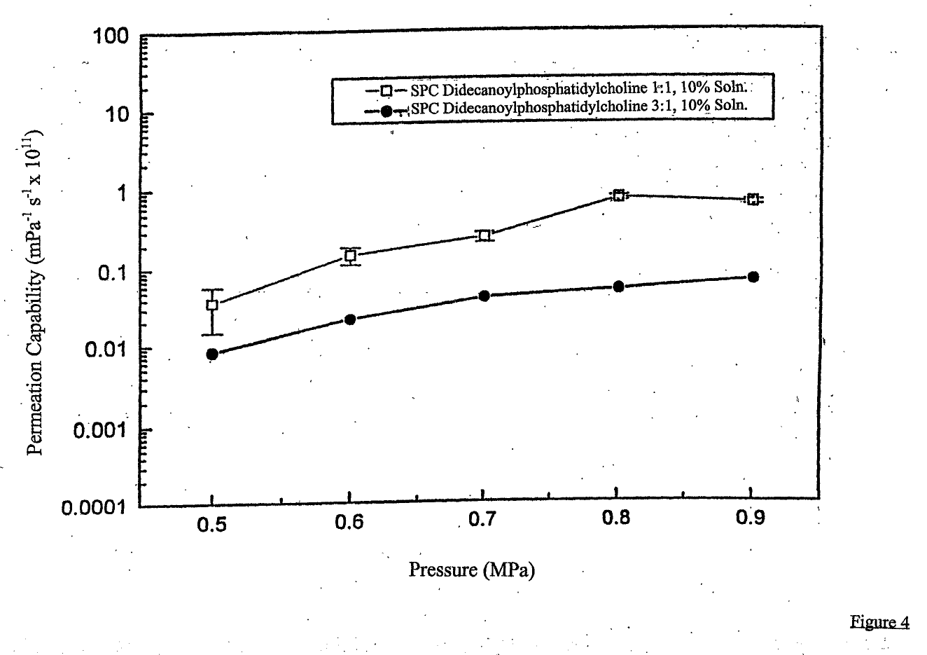

The carrier permeation capability is determined by the methods described in Examples 1 to 4. The corresponding results are shown in FIG. 4. They indicate that an addition of didecanoyl phosphatidyl choline significantly increases the permeation capability of the carrier as a function of the concentration, particularly at high pressures. The carriers, formed from SPC and didecanoyl phosphatidyl choline in a molar ratio of 1:1 (with the exception of the carriers with a molar ratio of 3:1), have a significantly higher permeation capability than do the liposomes formed from pure SPC.

The values of the permeation capability for the carriers of Examples 5 to 6, which were measured, are summarized in Table 2.

The 10% suspension, containing the pure didecanoyl phosphatidyl choline, is milky cloudy. This suspension contains carriers with an average diameter of 700±150 nm and forms a sediment. This behavior clearly shows that the lipid cannot be solubilized in the relevant concentration range either by itself or in combination with SPC.

TABLE 2

| | Pore Diameter | Pressure | Permeation |
|---|---|---|---|
| Sample Description | (nm) | (MPa) | Capability |
| Sample: 3:1 | 50 | 0.9 | 0.00039 |
| | 100 | 0.5 | 0.0083 |
| | | 0.6 | 0.021 |
| | | 0.7 | 0.04 |
| | | 0.8 | 0.05 |
| | | 0.9 | 0.066 |
| Sample: 1:1 | 50 | 0.9 | 0.16 |
| | 100 | 0.5 | 0.052 |
| | | | 0.021 |
| | | 0.6 | 0.12 |
| | | | 0.17 |
| | | 0.7 | 0.27 |
| | | | 0.22 |
| | | 0.8 | 0.76 |
| | | | 0.69 |
| | | 0.9 | 0.66 |
| | | | 0.60 |

### EXAMPLE 7

| | |
|---|---|
| 345.6 mg | phosphatidyl choline from soy beans |
| | (purer than 95%, PC) CMC = 10⁻⁷ M |
| 154.4 mg | distearoyl phosphomaltobionamide |
| | CMC = 10⁻⁵ M |
| 4.5 ml | buffer, pH 7.3 |

A suspension of SPC/DSPE maltobionamide is prepared in a molar ratio of 3:1 according to the method described for Examples 5 to 6. The resulting carriers have an exceptionally good permeation capability. For the determination of the permeation capability, the size of the carrier is determined before and after each measurement. The measurements prove that there is no solubilization of the carrier at any time.

The permeation capability of the carrier is determined at a pressure of 0.4 MPa and in, contrast to Examples 5 to 6, at a temperature of 52° C. At this pressure, the permeation of the carrier through the artificial permeation barrier is observed to be adequately good. The lipid added (glycolipid) is incapable of solubilizing the phospholipid. An investigation of the suspension by means of dynamic light scattering as well as by optical microscopy gives no indication of the existence of the solubilized (micellar) phase. The final size of the particles after permeation through the artificial permeability barrier depends on the driving pressure (0.3 to 0.9 MPa; as the pressure increases, the tendency decreases) and is between 98 and 81 nm.

Pure glycolipid does not dissolve or form a micellar suspension; instead, it forms a vesicle suspension. In order to prove this, an experiment was carried out, with which the osmotic activity of DSPE in the aqueous medium can be determined. For this purpose, the lipid suspension was diluted with water. Because of the thereby arising concentration gradient, water enters the vesicles. As a direct consequence, the average vesicle radius increases measurably. On the other hand, particles without an internal volume (such as mixed micelles), do not change their size under comparable experimental conditions.

### EXAMPLES 8-17

Composition:

| | |
|---|---|
| 203-86.5 μl | phosphatidyl choline from soy beans |
| | (as a 1:1 weight/volume SPC solution in absolute |
| | ethanol) CMC (in water) ∞ 10⁻⁷M |
| 9.04-61.4 mg | diclofenac, solubility ≦ 10^{-?}M |
| 1 ml | phosphate buffer (nominal): pH 6.5 |

The carriers are prepared as SPC/diclofenac mixtures in a molar ratio of 4:1 to 1:4 by the method described in Examples 1 to 4.

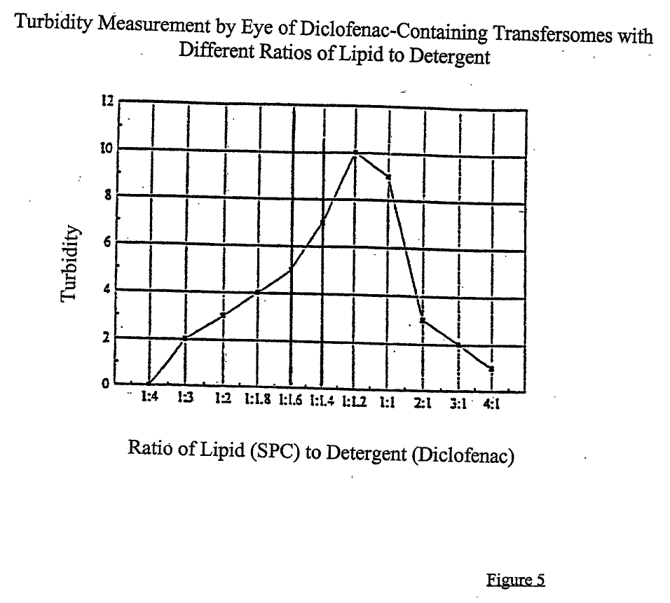

The mixtures so obtained are exposed to a source of ultrasound, until the samples are clear macroscopically (for approximately 4 minutes). After that, the solutions are centrifuged for 15 minutes at 15,000 rpm. The resulting 1:1 to 1:4 solutions are not clear (FIG. 5); instead they are opalescent. On the other hand, the 4:1, 3:1 and 2:1 mixtures show clear deposits. After being allowed to stand for 5 minutes, the other suspensions also become cloudy, a flaky precipitate being formed by the 1:2, 1:3 and 1:4 mixtures (Table 3). The preparations show this behavior even after the pH is adjusted with HCI to values between 7 and 7.2.

Determination of the Carrier Permeation Capability:

The carrier permeation capability, which is a measure of the carrier deformability, is determined as described in the preceding examples. For mixtures with 15 mg/ml, 20 mg/ml and 25 mg/ml of diclofenac, at a pressure of 0.3 MPa (driving pressure), the following permeability values (P) are obtained: 6×10⁻¹¹ m/Pa/s, 10⁻¹⁰ m/Pa/s and 2.5×10⁻¹⁰ m/Pa/s.

These values are comparable with those of known transfersomes, which were measured under similar conditions (SPC/NaChol 3/1 M/M; 2% by weight: 3×10⁻¹⁰ m/Pa/s). This proves that SPC/diclofenac mixtures of suitable composition have a very high permeation capability and consequently must be extremely deformable, although they cannot be solubilized at any time or any concentration.

TABLE 3
The pH is adjusted to a value between 7 and 7.2 with HCl and the mixture
is ultrasonicated.

| | | |
|---|---|---|
| After ultrasonication: | 1:1.0 | slightly cloudy |
| | 1:1.2 | cloudy, liquid, crystals in solution about |
| | | 20 per sight field |
| | 1:1.4 | cloudy, liquid, crystals in solution about |
| | | 20 per sight field |
| | 1:1.6 | cloudy, liquid, crystals somewhat larger |
| | 1:1.8 | cloudy, viscous, crystals ball together |
| | 1:2.0 | cloudy, viscous, very many crystals |
| | 1:2.2 | cloudy, viscous, very many very large |
| | | crystals |

### EXAMPLES 18-25:

Composition:

475-325 mg phosphatidyl choline from soy beans

| | |
|---|---|
| 475-325 mg | phosphatidyl choline from soy beans |
| | CMC ∞ 10⁻⁷M |
| 25-175 mg | ibuprofen, solubility ≦ 5 × 10⁻⁵M |
| 5 ml | buffer, pH 6.5 |

Preparation:

The preparation is as described in Examples 1 to 4, with the exception that, after the mixture is suspended, the pH is adjusted to a value of 7 by the addition of 10 M NaOH. In each case, 5 ml of ibuprofen-containing transfersomes are prepared with increasing amounts of ibuprofen and decreasing amounts of SPC (in 25 mg steps), the total lipid concentration being 10%.

Microscopic Check of the Suspensions Obtained:

Sample 1: no crystals, very large carriers,

Sample 2: no crystals, very large carriers,

Sample 3: only flickering in background,

Sample 4: small crystals very occasionally,

Sample 5: no crystals, droplets,

Sample 6: predominantly crystals,

Sample 7: droplets, isolated very large crystals.

Determination of the Carrier Permeation Capability:

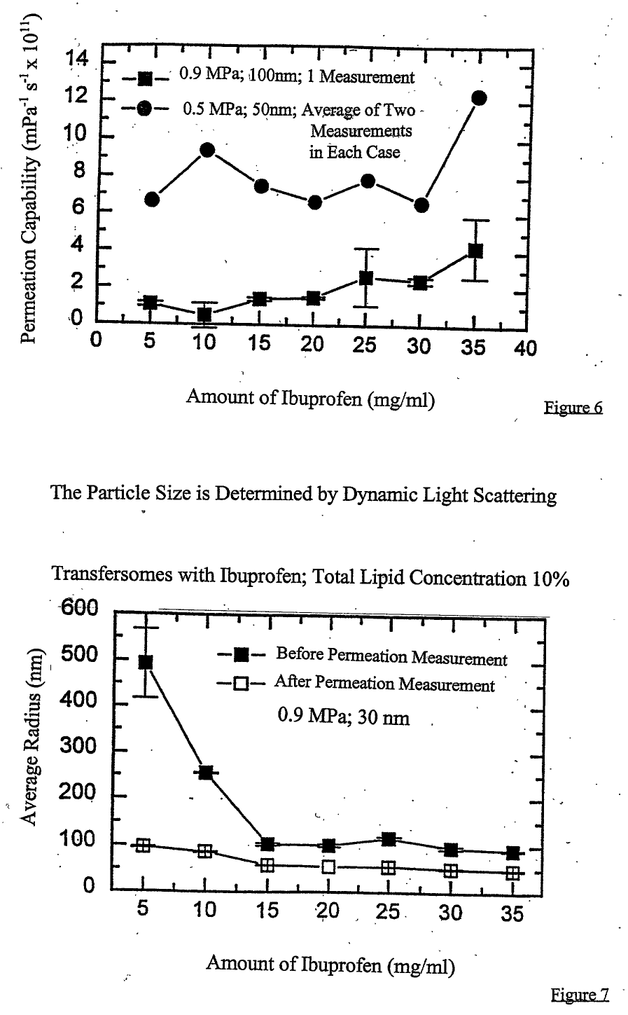

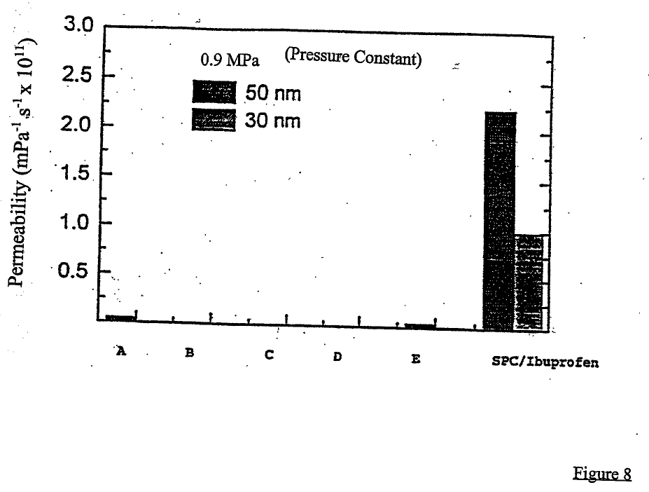

The determination of the carrier permeation capability is carried out as described in the preceding examples. The results of this measurement are shown in FIGS. 6 and 7. The mixtures of phospholipid and active ingredient investigated show typical transfersome behavior throughout, but particularly in the concentration range of 35 mg of ibuprofen per ml and above. The ibuprofen concentration of the carriers brings about no solubilization.

### COMPARISON EXAMPLES A-E

Comparison Example A (Example 2 of the EP-A 0 211 647)

Composition:

| | | |
|---|---|---|
| 120 | mg | dipalmitoyl phosphatidyl choline (DPPC) |
| 24 | mg | oleic acid |
| 20 | mg | arginine |
| 60 | ml | PBS (dissolve one tablet in 200 ml of distilled water) |

DPPC (120.0 mg) and 24.1 mg of oleic acid were weighed into a 100 ml beaker. Subsequently, the two reagents were mixed. A phosphate buffer salt (PBS) tablet was dissolved completely in 200 ml of distilled water in order to obtain a 10 mM (PBS) buffer. Arginine (20 mg) was then dissolved in 60 ml of PBS with a pH of 7.46 and added to the lipid mixture. The solution obtained was heated for 30 minutes at 40° to 45° C. and . . . (TRANSLATOR'S NOTE: German text is incomplete here).

Comparison Example B (Example 9 of the EP-A 0 280 492)

| | | |
|---|---|---|
| 270 | mg | dipalmitoyl phosphatidyl choline (DPPC) |
| 30 | mg | DSPC |
| 60 | mg | 1-octadecane sulfonic acid (ODS) |

DPPC (270.05 mg), 30.1 mg of DSPC and 60.1 mg of 1-octadecane sulfonic acid (ODS) were dissolved in 1:1 chloroform/methanol. The sample was evaporated to dryness for 2 hours in a rotary evaporator. Subsequently, drying was continued for a further hour under vacuum. The residue was rehydrated with 10 ml of PBS. The mixture was heated to 60° C. and homogenized. After that, the sample was exposed for 5 minutes to ultrasound.

Comparison Example C (Example 7 of the WO 88/07632):

| Composition: | |
|---|---|
| 400 mg | Setacin F special paste |
| | (disodium lauryl sulfosuccinate) |
| 580 mg | hydrogenated PC (PHPC) |
| 200 mg | Minoxidil |
| | acetate buffer pH 5.5 |

Setacin F special paste (400 mg), 580.03 mg of PHPC and 200.03 mg of Minoxidil were weighed into a beaker and dissolved in 1:1 chloroform/methanol and transferred to a round-bottom flask. The lipid mixture was concentrated for about 2.5 hours in a rotary evaporator and subsequently dried completely under vacuum. The sample was then shaken in a warm water bath at 50° C. and rehydrated with 10 ml of acetate buffer. After the sample has gone into solution completely, the solution is allowed to stand for 1 hour in the water bath shaker.

As antioxidant, 1 mg of deferoxamine-mesylate were added. The pH of the solution was then adjusted to a value of about 7.24 by the addition of 1 drop of 10 mM HCI. The solution could be homogenized macroscopically by stirring at a water bath temperature of 35° C.

Comparison Example D (Example 4 of the EP-A 0 220 797)

| Composition: | |
|---|---|
| 400 mg | purified hydrogenated soybean lecithin |
| 40 mg | HCO-60 (ethoxylated hydrogenated castor oil) |
| 100 mg | vitamin E |
| 9.46 ml | doubly distilled water |

Phospholipon 90 H (hydrogenated soybean lecithin, 400.04 mg), 40 mg of Emulgin HRE 60 (ethoxylated hydrogenated castor oil) and 100.11 mg of vitamin E were weighed into a 100 ml beaker and 9.46 ml of doubly distilled water were added. The sample was stirred for 45 minutes, until almost everything had dissolved. The lipid solution was then exposed for 10 minutes at 79° C. to ultrasound. To complete the dissolving, the sample was stirred once again and exposed to ultrasound for 10 minutes at 56° C.

Comparison Example E (Example 2 of the EP-A 0 102 324)

| Composition: | |
|---|---|
| 300 mg | SPC |
| 150 mg | octadecyltrimethylammonium bromide |
| 2550 μl | distilled water |

SPC (300 mg) and 150 mg of octadecyltrimethylammonium bromide were weighed into a 100 ml beaker and dissolved in 1 ml of 1:1 chloroform/methanol.

The sample was evaporated to dryness under vacuum. A 1% solution was prepared by the addition of distilled water. The solution obtained was stirred for 15 minutes.

Unless stated otherwise, samples of the Comparison Examples A to E were prepared in accordance with the directions given in the publications named.

In FIG. 8, the permeation capability (at a constant pressure of 0.9 MPa) is shown for the Comparison Examples A to E and for an inventive ibuprofen/SPC transfersome in the form of a bar graph. It is clearly evident from the bar graph (FIG. 8) that, at an elevated pressure (0.9 MPa), the permeation capability of the compositions of the Comparison Examples A to E is significantly less than that of the inventive transfersomes.

1. Preparations for the application, administration or transport of at least one active ingredient, especially for medicinal or biological purposes, into and through barriers and constrictions, such as skin or the like, in the form of liquid droplets, which can be suspended in a liquid medium and are provided with a membrane-like sheath of one or a few layers of amphiphilic carrier substance, the carrier substance comprising at least two physicochemically different components, characterized in that at least two components are provided, which differ in their solubility in the suspension medium of the preparations, usually water, by a factor of at least 10 and in that the content of solubilizing components is less than 0.1 mole percent, based on the content of these substances, at which the solubilization point of the enveloped droplets is reached or this solubilization point cannot be reached.

2. The preparation of claim 1, characterized in that the amphiphilic components are selected so that, independent of concentration, there is no solubilization.

3. The preparation of claims 1 and 2, characterized in that the solubility, especially the water solubility of the more soluble component(s) is/are at least 10⁻³ to 10⁻⁶ M and the solubility, especially the water solubility, of the less soluble component(s) is/are at least 10⁻⁶ to 10⁻¹⁰ M.

4. The preparation of one of the claims 1 to 3, characterized in that the difference between the solubility of the more soluble component(s) and the less soluble component(s) is approximately between 10 and 10⁷, preferably between 10² and 10⁶ and especially between 10³ and 10⁵.

5. The preparation of one of the claims 1 to 4, characterized in that the ability of the preparation to permeate through constrictions is at least 0.001% and preferably 0.1% of the permeability of small molecules, which permeate essentially without being impeded.

6. The preparation of one of the claims 1 to 5, characterized in that the ratio of the permeation capability relative to reference particles P_{(transfer.)}/P_{(refer.)}, the reference particles being, for example water, much smaller than the constrictions in the barrier, when the barrier itself is the site of the determination, is between 10⁻⁵ and 1, preferably between 10⁻⁴ and 1 and especially between 10⁻² and 1.

7. The preparation of one of the claims 1 to 6, characterized in that the preparations contain at least two amphiphilic components of different solubility, for forming a carrier substance and/or a membrane-like sheath about a droplet amount of hydrophilic liquid, wherein the active ingredient is contained in the carrier substance in or at the membrane-like sheath and/or in the hydrophilic liquid.

8. The preparation of one of the claims 1 to 7, characterized in that the vesicle radius of the
enveloped droplets is between about 25 and about 500 mn, preferably between about 50 and about 200 nm and especially between 80 and about 100 nm.

9. The preparation of one of the claims 1 to 8, characterized in that the sheath is a double layer.

10. The preparation of one of the claims 1 to 9, characterized in that the amphiphilic component (n) comprises or comprise physiologically tolerated lipids of different polarity and/or such an active ingredient or ingredients.

11. The preparation of one of the claims 1 to 10, characterized in that the amphiphilic substance comprises a lipid or lipoid of biological origin or a corresponding synthetic lipid or a derivative of such lipids, particularly diacyl or dialkyl glycerophosphoethanolamino azo polyoxyethylene derivative, didecanoyl phosphatidyl choline, diacyl phosphooligomaltobionamide, a glyceride, a glycerophospholipid, isoprenoid lipid, sphingolipid, steroid, sterol, a sulfur-containing or hydrocarbon-containing lipid or a different lipid, which forms stable structures, such as double layers, preferably comprises a half protonated liquid fatty acid, particularly a phosphatidyl choline, phosphatidyl ethanolamine, phosphatidyl glycerol, phosphatidyl inositol, a phosphatid acid, a phosphatidyl serine, a sphingomylein or sphingophospholipid, glycosphingolipid (such as cerebroside, ceramide polyhexoside, sulfatide, sphingoplasmalogen), ganglioside or other glycolipid, or a synthetic lipid, preferably a dioleoyl, dilinolyl, dilinolenyl, dilinoloyl, dilinolinoyl or diarachinoyl, dilauroyl, dimyristoyl, dilalmitoyl, distearoyl phospholipid or a corresponding dialkyl or sphingosin derivative, glycolipid or other identical chain or mixed chain acyl lipid or alkyl lipid.

12. The preparation of one of the claims 1 to 11, characterized in that the less soluble amphiphilic component comprises a synthetic lipid, preferably myristoleoyl, palmitoleoyl, petroselinyl, petroselaidyl, oleoyl, elaidyl, cis- or trans-vaccenoyl, linolyl, linolenyl, linolaidyl, octadecatetraenoyl, gondoyl, eicosaenoyl, eicosadienoyl, eicosatrienoyl, arachidoyl, cis- or trans-docosaenoyl, docosadienoyl, docosatrienoyl, docosatetraenoyl, caproyl, lauroyl, tridecanoyl, myristoyl, pentadecanoyl, palmitoyl, heptadecanoyl, stearoyl or nonadecanoyl, glycerophospholipid or a corresponding chain-branched derivative or a corresponding sphingosin derivative, glycolipid or a different acyl lipid or alkyl lipid; and the more soluble component or components is derived from one of the less soluble components listed above and, for increasing the solubility, is derivatized with a butanoyl, pentanoyl, hexanoyl, heptanoyl, octanoyl, nonanoyl, decanoyl, dodecane or undecanoyl or a corresponding monounsaturated or polyunsaturated or chain-branched substituent thereof or several substituents, selected independently of one another, and/or is substituted, complexed and/or associated with a different material, which is suitable for improving the solubility.

13. The preparation of one of the claims 1 to 12, characterized in that the total content of amphiphilic substance for administration on human or animal skin is between 0.01 and 40% by weight of the preparation, preferably between 0.1 and 15% by weight and especially between 1 and 10% by weight.

14. The preparation of one of the claims 1 to 13, characterized in that the total content of amphiphilic substance for application on plants is 0.000001 to 10% by weight, preferably between 0.001 and 1% by weight and especially between 0.01 and 0.1% by weight.

15. The preparation of one of the claims 1 to 14, characterized in that, as active ingredient, it contains an adrenocorticostatic agent, a β-adrenolytic agent, an androgen or antiandrogen, an anti-parasitic, anabolic, anesthetic or analgesic, analeptic, anti-allergic, anti-arrhythmic, anti-arteriosclerosis, anti-asthmatic and/or bronchospasmolytic agent, an antibiotic, an anti-depressive and/or anti-psychotic agent, an anti-diabetic agent, an antidote, an anti-emetic, anti-epileptic, anti-fibrinolytic, anti-convulsive or anti-cholinergic agent, an enzyme, coenzyme or a corresponding inhibitor, an antihistamine, an antihypertensive drug, a biological activity inhibitor, an antihypotensive agent, an anticoagulant, an anti-mycotic or antimyasthenic agent, an active ingredient against Parkinson's or Alzheimer's disease, an anti-phlogistic, anti-pyretic or anti-rheumatic agent, an antiseptic, a respiratory analeptic or stimulating agent, a broncholytic, cardiotonic or chemotherapeutic agent, a coronary dilator, a cytostatic agent, a diuretic, a ganglion blocker, a glucorcorticoid, a therapeutic agent for influenza, a hemostatic or hyptonic agent, immunoglobulin or fragment or a different immunological or receptor substance, a bioactive carbohydrate (derivative), a contraceptive, a migraine agent, a mineral corticoid, a morphine antagonist, a muscle relaxant, a narcotic, a neural or CNS therapeutic agent, a nucleotide or polynucleotide, a neuroleptic agent, a neuron transmitter or a corresponding antogonist, a peptide (derivative), an ophthalmic agent, a (para)-sympathicomimetic or (para)-sympathicolytic agent, a protein (derivative), a psoriasis/neurodermatitis agent, a mydriatic agent, a mood elevator, rhinological agent, a sleeping draft or its antagonist, a sedative, a spasmolytic, tuberculosis or urological agent, a vasoconstrictor or dilator, a virostatic agent or a wound-healing agent or several such agents, especially diclofenac or ibuprofen.

16. The preparation of one of the claims 1 to 15, characterized in that the active ingredient is a nonsteroidal anti-inflammatory drug, for example, diclofenac, ibuprofen or a lithium, sodium, potassium, cesium, rubidium, ammonium, monoethyl, dimethyl, trimethylammonium or ethylammonium salt thereof.

17. The preparation of one of the claims 1 to 16, characterized in that the less polar component comprises a physiologically compatible lipid, preferably from the class of phospholipids and especially from the class of phosphatidyl cholines, and the active ingredient is the more soluble component, optionally with the addition of less than 10% by weight, based on the total composition of the preparation, of a further soluble component, which is the more soluble component, the concentration of the more soluble component(s) typically being between 0.01% by weight and 15% by weight, preferably between 0.1% by weight and 10% by weight and particularly between 0.5% by weight and 3% by weight, and the total lipid concentration being between 0.005% by weight and 40% by weight and preferably between 0.5% by weight and 15% by weight and especially between 1% by weight and 10% by weight.

18. The preparation of one of the claims 1 to 17, characterized in that the preparation comprises consistency modifiers, such as hydrogels, antioxidants such as probucol, tocopherol, BHT, ascorbic acid, desferroxamine and/or stabilizers such as phenol, cresol, benzyl alcohol, etc.

19. The preparation of one of the claims 1 to 18, characterized in that the active ingredient is a growth regulating substance for living beings.

20. The preparation of one of the claims 1 to 18, characterized in that the active ingredient has biocidal properties and, in particular, is an insecticide, pesticide, herbicide or fungicide.

21. The preparation of one of the claims 1 to 18, characterized in that the active ingredient is an allurement, in particular, a pheromone.

22. A method for producing a preparation for the administration, application or transport of at least one active ingredient, particularly for medicinal or biological purposes, into and through natural barriers and constrictions, such as skin and the like, in the form of liquid droplets, which can be suspended in a liquid medium and are provided with a membrane-like sheath of one or a few layers of amphiphilic carrier substance, the carrier substance comprising at least two physicochemically different components, characterized in that at least two amphiphilic components are selected, which differ in their solubility in the suspension medium of the preparation, usually water, by a factor of at least 10 and the content of solubilizing components is less than 0.1 mole percent, based on the content of these substances, at which the solubilizing point of the enveloped droplets is reached or this point cannot be reached in a practically relevant region, and the content of amphiphilic components is adjusted, so that the ability of the preparation to permeate through constructions is at least 0.001% of the permeability of small molecules, for example, of water.

23. The method of claim 22, characterized in that the content of amphiphilic components is adjusted, so that the ratio of the permeation capability relative to reference particles, which are much smaller than the constrictions in the barrier, for example water, when the barrier itself is the site of determination, is between 10⁻⁵ and 1, preferably between 10⁻⁴ and 1 and especially between 10⁻² and 1.

24. The method of claims 22 and 23, characterized in that stability and permeation capability are determined by filtration, optionally under pressure, through a fine-pored filter or through otherwise controlled mechanical whirling up, shearing or comminuting.

25. The method of one of the claims 22 to 24, characterized in that the substance mixture for producing a transfersome-like preparation is subjected to a filtration, to a treatment with ultrasound, to stirring, to shaking or to other mechanical comminuting effects.

26. The method of one of the claims 22 to 25, characterized in that transfersome-like droplets, which form the preparation, are produced from at least two amphiphilic components of different polarity, at least one polar liquid and at least one active ingredient.

27. The method of one of the claims 22 to 26, characterized in that the transfersome-like droplets, which form the preparation, wherein the amphiphilic component(s) comprises or contains the active ingredient, are formed from at least two amphiphilic components of different polarity and at least one polar liquid.

28. The method of one of the claims 22 to 28, characterized in that the amphiphilic components and the hydrophilic substance in each case are mixed separately with the active ingredient and optionally brought into solution, the mixtures or solutions are then combined into a mixture, in which droplet formation is brought about by supplying, in particular, mechanical energy.

29. The method of one of the claims 22 to 28, characterized in that the amphiphilic components, either as such or dissolved in a physiologically compatible solvent or dissolving intermediary, which is miscible with a polar liquid or liquids, especially with water, are combined with a polar solution.

30. The method of one of the claims 22 to 29, characterized in that the formation of enveloped droplets is brought about by stirring, by evaporation from a reverse phase, by an injection method or a dialysis method, by electrical, thermal or mechanical stressing, such as shaking, stirring, homogenizing, ultrasonicating, rubbing, freezing or thawing, heating or cooling or high pressure or low pressure filtration.

31. The method of one of the claims 22 to 30, characterized in that the formation of the enveloped droplets is brought about by filtration and the filter material has a pore size of 0.01 to 0.8 μm, especially of 0.05 to 0.3 μm and particularly of 0.08 to 0.15 μm, several filters optionally being connected in series.

32. The method of one of the claims 22 to 31, characterized in that the association between carrier and active ingredients takes place at least partially after the droplet formation.

33. The method of one of the claims 22 to 32, characterized in that, shortly before use, the enveloped droplets are prepared from a concentrate or lyophilisate.
